(19) United States
(12) Patent Application Publication
(10) Pub. No.: US 2015/0238460 A1
(43) Pub. Date: Aug. 27, 2015

(54) Methods and Compositions For Treating Hepatocellular Carcinoma

(71) Applicant: GENSPERA, INC, SAN ANTONIO, TX (US)

(72) Inventor: Devalingam Mahalingam, San Antonio, TX (US)

(73) Assignee: GENSPERA, INC., SAN ANTONIO, TX (US)

(21) Appl. No.: 14/423,398

(22) PCT Filed: Aug. 23, 2013
(86) PCT No.: PCT/US13/56523

§ 371 Date: Feb. 23, 2015

Related U.S. Application Data
(60) Provisional application No. 61/693,273, filed on Aug. 25, 2012.

(51) Int. Cl. A61K 31/365; A61K 45/06; A61K 47/48

(52) U.S. Cl. CPC A61K 31/365; A61K 47/48315; A61K 45/06

(57) ABSTRACT

Provided herein are methods of treating subjects having tumors. For example, the invention relates to a method for treating subjects having hepatocellular carcinoma by administering an effective amount of a therapeutic prodrug.

### PRIORITY

This Application claims priority to U.S. Provisional Appln. Ser. No. 61/693,273, filed Aug. 25, 2012, and PCT/US13/56523 filed Aug. 23, 2013, both of which are herein incorporated by reference in their entirety.

### FIELD OF THE INVENTION

The invention relates to methods of treating subjects having epithelial tumors. More specifically, the invention relates to a method for treating subjects having hepatocellular carcinoma by administering an effective amount of a therapeutic prodrug.

### BACKGROUND OF THE INVENTION

Pro-drug chemotherapy is an approach to cancer treatment that is being investigated as a means to achieve higher concentrations of cytotoxic or biologically active agents at a tumor location while avoiding systemic toxicity. With pro-drug chemotherapy, a relatively non-toxic form of a cytotoxin, the pro-drug, is converted into the active cytotoxic agent at the tumor site or other specific location. G-202, as defined herein, is a thapsigargin prodrug; it consists of a cytotoxic analog of thapsigargin coupled to a masking peptide which inhibits its biologic activity until proteolytic cleavage at the tumor site. Thapsigargin is a natural product with profound effects on cell viability. Thapsigargin is a non-cell-type specific toxin with documented ability to kill a broad spectrum of cancer cell lines as well as normal endothelial cells, fibroblasts and osteoblasts. It induces a rapid and pronounced increase in the concentration of cytosolic calcium, due to blockade of the Sarcoplasmic/Endoplasmic Reticulum Calcium ATPase (SERCA) pump to which it binds with high affinity. See, e.g., Denmeade, S. R., et al., J Natl Cancer Inst 95: 990-1000, 2003; Davidson, G. A., et al., J Biol Chem 270: 11731-11734, 1995; Furuya, Y., et al., Cancer Res 54: 6167-6175, 1994; Denmeade, S. R., et al., Prostate 28: 251-265, 1996; Tombal, B. et al., Cell Calcium 25: 19-28, 1999; Tombal, B. et al., Prostate 43: 303-317, 2000. The increase in cytosolic calcium leads to induction of apoptosis and ensuing cell death.

The masking peptide component of G-202 is a substrate for Prostate-Specific Membrane Antigen (PSMA). PSMA is a glutamate carboxypeptidase type II that cleaves the acidic amino acids glutamate (Glu) and aspartate (Asp). G-202 is produced by coupling 8-O-(12-aminododecanoyl)-debutanoyl thapsigargin (12ADT) to the beta carboxyl of Asp at the N terminal end of the masking peptide Asp-Glu-γ-Glu-γ-Glu-γ-Glu (wherein the hyphen denotes alpha linkage and gamma symbol denotes gamma linkage) to produce the prodrug 12ADT-β-Asp-α-Glu-γ-Glu-γ-Glu-γGluOH. (i.e., G-202). Without being bound to any particular theory, it is believed that PSMA sequentially hydrolyzes the Glu residues to release the active cytotoxin 12ADT-Asp. Because PSMA is expressed on the surface of prostate cancer cells and on the surface of endothelial cells within most solid tumors, but not in normal tissues and not on endothelial cells outside of most solid tumors, release of the cytotoxin 12ADT-Asp is expected to be primarily confined to tumor tissue. See, e.g., Wright, G. L., et al., Urol Oncol 1:18-28, 1995; Lopes, A. D., et al., Cancer Res 50:6423-6429, 1990; Silver, D. A., et al., Clin Cancer Res 3:81-85, 1997; Chang, S. S., et al., Cancer Res 59:3192-3198, 1999; Israeli, R. S., et al., Cancer Res 54:1807-1811, 1994; Cunha, A. C., et al., Cancer Lett. 236:229-38, 2006; Chang, S. S., et al., Clin Cancer Res 5:2674-2681, 1999; U.S. Pat. Nos. 7,767,648and 7,468,354.

Hepatocellular carcinoma (HCC) is the fifth most common tumor worldwide, and ranks as the third most common cause of death from cancer due to its poor prognosis (American Cancer Society 2007). More than 75% of cases occur in the Asia-Pacific region, largely in association with chronic hepatitis B virus (HBV) infection. See, e.g., Cheng, A. L., et al. Lancet Oncol, 2009;10:25-34; Llovet, J. M., et al., Lancet, 2003;362:1907-17. An estimated 360,000 patients residing in East Asian countries, including China, Japan, Korea, and Taiwan, die from this disease each year. See, e.g., El-Serag, H. B. et al., J Clin Gastroenterol, 2002; 35(5 Suppl 2):572-8. Over the next two decades the incidence of HCC is expected to rise in the United States (U.S.) reflecting, in part, the current Hepatitis C epidemic. In the U.S., HCC currently has the second highest increase in incidence and the highest increase in death rates of any tumor in the last 10 years (see, e.g., Hussain, S. A., et al. Ann Oncol, 2001;12:161-72).

Most HCC cases develop in the presence of chronic liver disease or cirrhosis. Unresectable HCC is an aggressive disease with a median survival at diagnosis of 6 months for untreated patients at advanced stages, and 16 to 20 months for intermediate disease (see, e.g., Llovet, J. M., et al., J Hepatol, 2008; 48 Suppl 1:520-37). This relatively low median survival is influenced by the fact that most HCC cases are diagnosed at an advanced stage of the disease; thus, at the time of diagnosis, surgical resection may be suitable only for approximately 5% of patients.

One current treatment for HCC is sorafenib (Nexavar®), a kinase inhibitor that targets intracellular Raf serine/threonine kinase isoforms including Raf-1 (or C-Raf), wild-type B-Raf, and mutant B-Raf. It also inhibits cell surface kinases such as stem cell factor receptor (KIT), FMS-like tyrosine kinase3 (FLT3), RET (a tyrosine kinase rearranged during transfection), VEGFR-1, VEGFR-2, VEGFR-3, and PDGFRβ. Sorafenib has received worldwide approval for the treatment of unresectable HCC. The approval of sorafenib provided the first effective antiangiogenic therapy for advanced HCC and is still the only approved treatment for this disease.

The efficacy of sorafenib in patients with advanced hepatocellular carcinoma

(HCC) has been evaluated in two randomized, double-blind, multicenter, Phase III trials: the SHARP Trial (Randomized Phase III Study Comparing Sorafenib vs. Placebo in Patients with Advanced Hepatocellular Carcinoma Who Had Not Received Prior Systemic Treatment) and a trial conducted in patients from the Asian-Pacific region. See, e.g., Cheng, A. L., et al., Lancet Oncol, 2009; 10:25-34; Llovet, J. M., et al., N Engl J Med, 2008; 359:378-90. In the SHARP Trial the time to radiologic progression was 5.5 months for sorafenib vs. 2.8 months for placebo, and overall survival improved from 7.9 months for placebo to 10.7 months for sorafenib. In the Asian-Pacific trial the results were less impressive with only a 6.5 months median overall survival vs. 6.5 months for placebo, and 2.8 months time to radiologic progression compared to 1.4 months for placebo. Thus, while these trials demonstrated a small survival benefit for the use of sorafenib as first line therapy for HCC, more effective therapies are urgently needed.

### SUMMARY OF THE INVENTION

In one aspect, the invention provides a method for treating subjects having epithelial tumors comprising administering the composition described herein in a therapeutically effective amount to a subject having an epithelial tumor. Epithelial tumors are known to those of ordinary skill in the art and include, for example, benign and premalignant epithelial tumors, such as breast fibroadenoma and colon adenoma, and malignant epithelial tumors, for example, prostate carcinoma. Malignant epithelial tumors include primary tumors, also referred to as carcinomas, and secondary tumors, also referred to as metastases of epithelial origin. More particularly, the invention provides a composition and method for treating subjects having hepatocellular carcinoma (also called hepatoma, malignant hepatoma and hepatocarcinoma).

Provided herein, according to another aspect of the invention are methods of treating a cell proliferative disorder, comprising administering the composition described herein in a therapeutically effective amount to a subject having the cell proliferative disorder.

In one embodiment, the disorder is benign. In another embodiment, the disorder is malignant. In another embodiment, the disorder is malignant and is an epithelial cancer, for example, prostate carcinoma or hepatocellular carcinoma.

In one embodiment, the composition is administered to a patient for at least one day of a 28-day cycle, which may be repeated multiple times. In this embodiment, the composition is administered at at least about 35 mg/m²; alternatively at at least about 45 mg/m²; alternatively at at least about 60 mg/m².

In one embodiment, the composition is administered to a patient for at least two consecutive days of a 28-day cycle, which may be repeated multiple times. In another embodiment, the composition is administered to a patient for at least 3 consecutive days of a 28-day cycle, which may be repeated multiple times. In alternative embodiments, the composition may be administered for additional consecutive or non-consecutive days within the 28-day cycle, at the same or varying doses.

In one embodiment, the composition is administered to a patient for at least two consecutive days of a 28-day cycle, which may be repeated multiple times. In this embodiment, on day 1 of each cycle the composition is administered at about 35 mg/m² to about 45 mg/m². In this embodiment, the dose of the composition administered on day 2 of each cycle is about 60 mg/m² to about 70 mg/m².

In another embodiment, the composition is administered to a patient for at least 3 consecutive days of a 28-day cycle, which may be repeated multiple times. In this embodiment, on day 1 of each cycle the composition is administered at about 35 mg/m² to about 45 mg/m². In this embodiment, the dose of the composition administered on day 2 of each cycle is about 60 mg/m² to about 70 mg/m². In this embodiment, the dose of the composition on day 3 of each cycle is about 60 mg/m² to about 70 mg/m².

In one embodiment, the composition is administered to a patient for at least one day of a 28-day cycle, which may be repeated multiple times. In this embodiment, on day 1 of each cycle the composition is administered at about 40 mg/m². In a further variation of this embodiment, the composition is administered to a patient for at least two consecutive days of a 28-day cycle, which may be repeated multiple times. In this embodiment, on day 2 of each cycle the composition is administered at about 66.8 mg/m². In yet a further variation of this embodiment, the composition is administered to a patient for at least 3 consecutive days of a 28-day cycle, which may be repeated multiple times. In this embodiment, the dose of the composition administered on day 3 of each cycle is about 66.8 mg/m².

In yet another alternative embodiment, the composition is administered to a patient for at least one day of a 28-day cycle, which may be repeated multiple times. In this embodiment, on day 1 of each cycle the composition is administered at about 40 mg/m². In a further variation of this embodiment, the composition is administered to a patient for at least two consecutive days of a 28-day cycle, which may be repeated multiple times. In this embodiment, on day 2 of each cycle the composition is administered at about 40 mg/m². In yet a further variation of this embodiment, the composition is administered to a patient for at least 3 consecutive days of a 28-day cycle, which may be repeated multiple times. In this embodiment, the dose of the composition administered on day 3 of each cycle is about 40 mg/m².

In a preferred embodiment of the invention, the composition is administered to a patient having hepatocellular carcinoma for at least one day of a 28-day cycle, which may be repeated multiple times. In another preferred embodiment of the invention, the composition is administered to a patient having hepatocellular carcinoma for at least two consecutive days of a 28-day cycle, which may be repeated multiple times. In another preferred embodiment of the invention, the composition is administered to a patient having hepatocellular carcinoma for at least three consecutive days of a 28-day cycle, which may be repeated multiple times.

In another preferred embodiment of the invention, the composition is administered to a patient having hepatocellular carcinoma for at least one day of a 28-day cycle, which may be repeated multiple times. In this embodiment, on day 1 of each cycle the composition is administered at about 40 mg/m². In a further variation of this embodiment of the invention, the composition is administered to a patient having hepatocellular carcinoma for at least two consecutive days of a 28-day cycle, which may be repeated multiple times. In this embodiment, on day 2 of each cycle the composition is administered at about 40 mg/m² or at about 66.8 mg/m². In yet a further variation of this embodiment of the invention, the composition is administered to a patient having hepatocellular carcinoma for at least three consecutive days of a 28-day cycle, which may be repeated multiple times. In this embodiment, the dose of the composition administered on day 3 of each cycle is about 40 mg/m² or at about 66.8 mg/m².

In one embodiment of the invention, the patient shows no progression of the carcinoma after treatment with the composition of the invention. In another embodiment, the patient shows a reduction in at least one symptom associated with a carcinoma after treatment with the composition of the present invention. In yet another embodiment, the patient shows improvement (i.e., either a reduction or an increase, as applicable) in a tumor marker associated with a carcinoma after treatment with the composition of the present invention. In another embodiment, the patient shows improvement (i.e., either a reduction or an increase, as applicable) in a tumor marker associated with a carcinoma and a reduction in at least one symptom associated with a carcinoma after treatment with the composition of the present invention.

In a preferred embodiment, the patient shows no progression of hepatocellular carcinoma after treatment with the composition of the invention. In this embodiment, the patient may also show a reduction in at least one symptom associated with hepatocellular carcinoma, or the patient may show improvement (i.e., either a reduction or an increase, as applicable) in a tumor marker associated with hepatocellular carcinoma after treatment with the composition of the present invention. In another embodiment, the patient shows no progression of hepatocellular carcinoma and shows improvement (i.e., either a reduction or an increase, as applicable) in a tumor marker associated with hepatocellular carcinoma and, further, shows a reduction in at least one symptom associated with hepatocellular carcinoma after treatment with the composition of the present invention.

Other advantages of the present invention will be apparent to one of skill in the art based on the present disclosure.

### DRAWINGS

FIG. 1 is a schematic drawing showing sequential PSMA hydrolysis of the 12ADT-Asp-Glu-γ-Glu-γ-Glu-γ-GluOH prodrug, G-202.

FIG. 2 shows PSMA Staining in HCC Tumor Vasculature.

In FIG. 2A, Tissue microarrays were stained for PSMA expression using the clone 3E6 anti-PSMA antibody. EC staining was graded on a zero- to three-point scale (table S1). Samples that contained any PSMA staining (that is, 1 to 3+) were considered to be positive. HCC, hepatocellular cancer; Mes, mesothelioma; OC, ovarian cancer; RCC, renal cell cancer; BrC, breast cancer; Mel, melanoma; BC, bladder cancer; NL, normal liver; NK, normal kidney; NBr, normal breast; NB, normal bladder; N, number of samples shown in parentheses.

FIG. 2B is an Example of 3+ PSMA staining of ECs (brown) in a 40 Å˜ section of hepatocellular carcinoma. Examples of brown EC staining indicated by arrows.

### DETAILED DESCRIPTION OF THE INVENTION

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

As used herein, the term “prostate specific membrane antigen” (PSMA) means prostate specific membrane antigen, as well as all other proteases that have the same or substantially the same proteolytic cleavage specificity as prostate specific membrane antigen.

As used herein, “Hepatocellular carcinoma”, “HCC”, and “malignant hepatoma” are used interchangeably and refer to primary and secondary (metastasized) tumors that originated from the liver tissue.

The terms “treat” or “treatment” refer to both therapeutic and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The terms “treating”, “treat”, or “treatment” embrace both preventative, e.g., prophylactic, and palliative treatment.

As used herein, “stable disease” means the stabilization of the disease state, for example, the stopping, delay or slowing of disease progression, amelioration or palliation of the disease state, and remission (whether partial or total), whether detectable or undetectable.

As used herein, “progressive disease” or “disease progression” means the worsening of the disease state, for example, the development of additional tumors, increase in size of a tumor, or the spread of the disease to other areas, including other organs.

The phrases “therapeutically effective amount” and “therapeutically effective dose” mean an amount of a compound of the present invention that (i) treats or prevents the particular disease, condition, or disorder, (ii) attenuates, ameliorates, or eliminates one or more symptoms of the particular disease, condition, or disorder, or (iii) prevents, reduces or delays the onset of one or more symptoms of the particular disease, condition, or disorder described herein. The reduction need not be complete. That is, a partial reduction in the symptom is contemplated. Additionally, the symptom need not be reduced permanently. A temporary reduction in at least one symptom is contemplated by the present invention.

In the case of cancer, the therapeutically effective amount of the drug may reduce the number of cancer cells; reduce the tumor size; inhibit (e.g., slow to some extent and preferably stop) cancer cell infiltration into peripheral organs; inhibit (e.g., slow to some extent and preferably stop) tumor metastasis; inhibit, to some extent, tumor growth; show improvement in biological markers associated with the cancer; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include epithelial cancers and other cancers described herein.

The term “prodrug” as used in this application refers to a precursor or derivative form of a pharmaceutically active substance that is less cytotoxic to tumor cells compared to the parent drug and is capable of being enzymatically or hydrolytically activated or converted into the more active parent form.

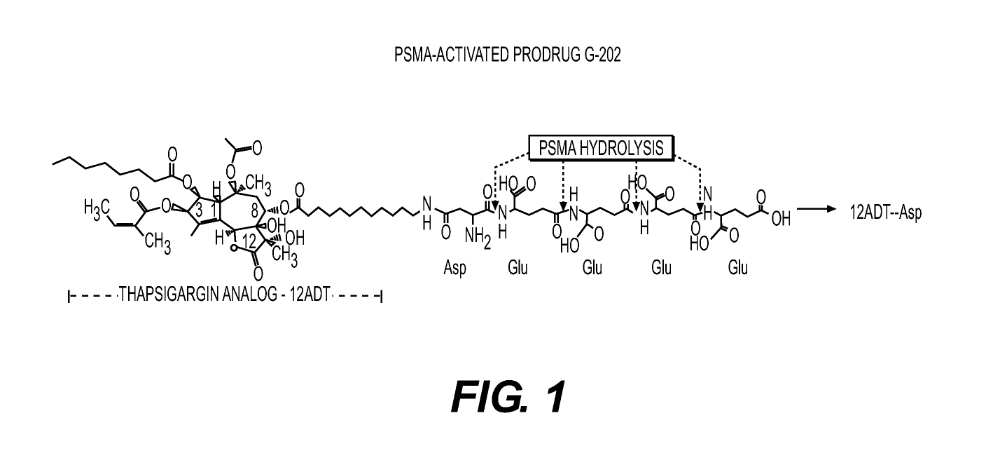

As used herein, the term “G-202” refers to the thapsigargin derivative 8-O-(12-aminododecanoyl)-debutanoyl thapsigargin (12ADT) linked to the aspartic acid of a peptide having the sequence Asp-Glu-γ-Glu-γ-Glu-γ-Glu, having the chemical structure shown in FIG. 1.

G-202 is a thapsigargin prodrug containing a cytotoxic analog of thapsigargin coupled to a masking peptide that inhibits its biologic activity until proteolytic cleavage at the tumor site. Thapsigargin itself is a natural product that is chemically modified to 8-O-(12-aminododecanoyl)-debutanoyl thapsigargin (12ADT). This thapsigargin analog is coupled to the beta carboxyl of Asp at the N-terminal end of the masking peptide Asp-Glu-γ-Glu-γ-Glu-γ-Glu to produce the prodrug 12ADT-Asp-Glu-γ-Glu-γ-Glu-γ-GluOH (G-202).

The chemical name for G-202 is 8-O-(12-aminododecanoyl)-debutanoyl-thapsigargin aspartate-glutamate-γ-glutamate-γ-glutamate-γ-glutamate OH. It is sometimes referred to in an abbreviated fashion: 12ADT-Asp-Glu-γ-Glu-γ-Glu-γ-GluOH, where 12ADT represents the thapsigargin derivative and Asp-Glu-γ-Glu-γ-Glu-γ-GluOH represents the PSMA-cleavable masking peptide. G-202 is a tan to white solid with a molecular weight of 1409.52.

The term “subject” refers to a mammal, including a human, which is to be the recipient of a particular treatment, or from whom cancer stem cells are harvested. Typically, the terms “subject” and “patient” are used interchangeably, unless indicated otherwise herein.

As used herein, the term “subject suspected of having cancer” refers to a subject that presents one or more signs or symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests.

The term “subject at risk for cancer” is a person or patient having an increased chance of cancer (relative to the general population). Such subjects may, for example, be from families with a history of cancer. Additionally, subjects at risk may be individuals in which there is a genetic history of a particular cancer associated with race, nationality or heritage or exposure to an environmental trigger.

As used herein, the term “administration” refers to the act of giving a drug, prodrug, or other agent, or therapeutic treatment (e.g., G-202) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

In a preferred embodiment of the present invention, G-202 is administered in a therapeutically effective amount to a patient at risk for cancer, a patient diagnosed with a cancer, or a patient suspected of having cancer. In one embodiment, G-202 is administered to such a patient for at least one day of a 28-day cycle, which may be repeated multiple times. In this embodiment, on day 1 of each cycle G-202 is administered at about 40 mg/m². In a further variation of this embodiment, G-202 is administered to such a patient for at least two consecutive days of a 28-day cycle, which may be repeated multiple times. In this embodiment, on day 2 of each cycle G-202 is administered at about 66.8 mg/m². In yet a further variation of this embodiment, G-202 is administered to such a patient for at least 3 consecutive days of a 28-day cycle, which may be repeated multiple times. In this embodiment, the dose of G-202 administered on day 3 of each cycle is about 66.8 mg/m². In an alternative of this embodiment, various amounts of G-202 (for example, 40 mg/m² or 66.8 mg/m²) may continued to be administered to such patient on days 4, 5, 6 or any other days of said 28-day cycle. Further, in each of these embodiments, the patient preferably has been diagnosed with or is suspected of having hepatocellular carcinoma or prostate carcinoma.

In an alternative of the preferred embodiment presented above, G-202 is administered in a therapeutically effective amount to a patient at risk for cancer, a patient diagnosed with a cancer, or a patient suspected of having cancer. In one embodiment, G-202 is administered to such a patient for at least one day of a 28-day cycle, which may be repeated multiple times. In this embodiment, on day 1 of each cycle G-202 is administered at about 40 mg/m². In a further variation of this embodiment, G-202 is administered to such a patient for at least two consecutive days of a 28-day cycle, which may be repeated multiple times. In this embodiment, on day 2 of each cycle G-202 is administered at about 40 mg/m². In yet a further variation of this embodiment, G-202 is administered to such a patient for at least 3 consecutive days of a 28-day cycle, which may be repeated multiple times. In this embodiment, the dose of G-202 administered on day 3 of each cycle is about 40 mg/m². In an alternative of this embodiment, various amounts of G-202 (for example, 40 mg/m² or 66.8 mg/m²) may continued to be administered to such patient on days 4, 5, 6 or any other days of said 28-day cycle. Further, in each of these embodiments, the patient preferably has been diagnosed with or is suspected of having hepatocellular carcinoma or prostate carcinoma.

Pharmaceutical formulations of the compounds of the invention, for example, G-202, can be prepared for oral, intravenous, or aerosol administration. In a preferred embodiment, G-202 is administered as an infusion. In this embodiment, G-202 may further be lyophilized for storage and transport, and is reconstituted to form a pharmaceutically acceptable formulation for administration as an infusion.

In an alternative embodiment, G-202 is administered as an injectable emulsion. In this embodiment, G-202 may further be lyophilized for storage and transport and is reconstituted to form a pharmaceutically acceptable formulation for administration as an injection.

Pharmaceutical formulations of G-202 preferably have the desired degree of purity and are optionally mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, e.g., carriers that are non-toxic to recipients at the dosages and concentrations employed.

The therapeutic compositions of the present invention can be packaged in suitably sterilized bottles or vials, either in multi-dose or in unit dose forms. The containers are preferably hermetically sealed after being filled with a composition of the invention. Preferably, the compositions are packaged in a container having a label affixed thereto, which label identifies the drugs present in the composition, and bears a notice in a form prescribed by a government agency such as the United States Food and Drug Administration, reflecting approval of the composition under appropriate laws, dosage information, and the like. The label preferably contains information about the composition that is useful to a health care professional administering the composition to a patient. The package also preferably contains printed informational materials relating to the administration of the composition, instructions, indications, and any necessary required warnings.

G-202

G-202 consists of a PSMA-selective 5 amino acid peptide substrate coupled to a highly cytotoxic analog of the natural product thapsigargin. See, e.g., Denmeade, S. R., et al., J. Natl. Cancer Inst. 2003; 9: 990-1000; and U.S. Pat. Nos. 7,767,648 and 7,468,354. Thapsigargin is isolated from the seeds of the plant Thapsia garganica, which grows as a weed throughout the Mediterranean basin. See, e.g., Rasmussen, U., et al., Acta Pharm. Suec. 1978; 15:133-140. Thapsigargin functions by potently inhibiting a critical intracellular protein, the sarcoplasmic/endoplasmic reticulum calcium ATPase (SERCA) pump whose normal function is to maintain intracellular calcium homeostasis in all cell types. Proper function of the SERCA pump is required for the viability of all cell types. Thus, thapsigargin inhibition of the SERCA pump results in the death of all cell types tested, both normal and malignant. See, e.g., Denmeade, S. R., et al., Sci. Transl Med. 2012; 4, 140ra86; Denmeade, S. R., et al., J. Natl. Cancer Inst. 2003; 9:990-1000; Pinto, J. T., et al., Clin. Cancer Res. 1996; 2:1445-1451.

On this basis, the prodrug of the present invention was designed to target this potent cytotoxin with a unique mechanism of action for selective activation by PSMA produced by prostate cancer epithelial cells within sites of prostate cancer and by tumor endothelial cells in other cancer cell types, for example, hepatocellular carcinoma. Without being bound by any particular theory, it is believed that PSMA is an extracellular carboxypeptidase that sequentially cleaves off acidic amino acids from the G-202 prodrug to eventually liberate a cytotoxic analog of thapsigargin. See, e.g., Pinto, J. T., et al., Clin. Cancer Res. 1996; 2:1445-1451; Carter, R. F., et al., Proc. Natl. Acad. Sci., USA 1996; 93:749-753; Mhaka, A., et al., Cancer Biol Ther. 2004; 3:551-558. This highly lipophilic analog, termed 12ADT-Asp, upon release from its water soluble peptide carrier, rapidly partitions into the surrounding cell membranes. See, e.g., Jakobsen, C. M., et al., J. Med. Chem. 2001; 44:4696-4703. The analog then binds to the SERCA pump producing a sustained elevation in intracellular calcium which results in activation of apoptosis (see, e.g., FIG. 1; Denmeade, S. R., et al. J. Natl. Cancer Inst. 2003; 9: 990-1000; Singh, P., et al., J. Med. Chem. 48, 3005-3014 (2005)). Because the 12ADT-Asp analog is released extracellularly into the tumor microenvironment, every cell does not need to produce PSMA to be killed by the prodrug activation. A substantial bystander effect is achieved by the release of the active drug into the tumor microenvironment.

Preclinical studies with G-202 have demonstrated that the prodrug is selectively activated by PSMA in vitro and is ˜60-fold more toxic to PSMA expressing vs. PSMA non-expressing tumor cells. PSMA shows significant growth inhibition against a panel of prostate, breast, renal, and bladder cancers in vivo at doses that are minimally toxic to the host animal. See, e.g., Denmeade, S., et al., www.ScienceTranslationalMedicine.org, Vol. 4, Issue 140: 1-12 (2012).

PMSA Expression in HCC

HCC is a highly vascularized tumor (see, e.g., Llovet, J. M., et al. N Engl J Med, 2008; 359:378-90). Tumor angiogenesis may be essential to its growth, invasion, or metastasis. See, e.g., Sergio, A, et al., Am J Gastroenterol. 2008; 103:914-21; Yamaguchi, R., et al., Hepatology. 1998; 28:68-77; Chao, Y., et al., Ann Surg Oncol. 2003; 10:355-62. Angiogenic factors such as angiopoietin, vascular endothelial growth factor (VEGF), platelet-derived growth factor (PDGF), and fibroblast growth factor-2 (FGF2), released from the tumor itself, inflammatory cells, and/or tumor stromal cells participate in the neovascularization of HCC. See, e.g., Miura, H. et al., J Hepatol. 1997; 27:854-61; Poon, R. T., et al., Ann Surg. 2001; 233:227-35; Torimura, T., et al., Hum Pathol. 1998 September; 29(9):986-91. It is this vascularity of HCC that often in clinical practice leads to a radiological (without tissue) diagnosis of HCC using dynamic phase imaging, e.g., “triple phase” or “liver protocol” computed tomography. HCC shows contrast enhancement in the arterial phase and “washout” of contrast media in the portal venous phase. Based on this high degree of vascularization, HCC may be particularly sensitive to therapies targeting the tumor vasculature.

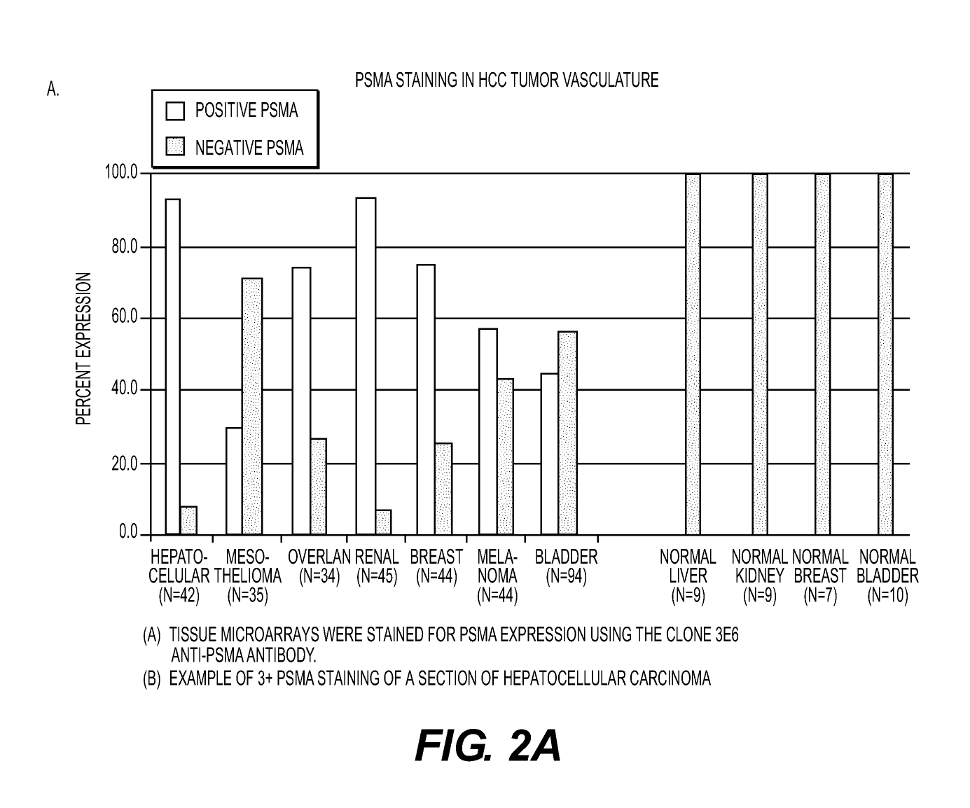

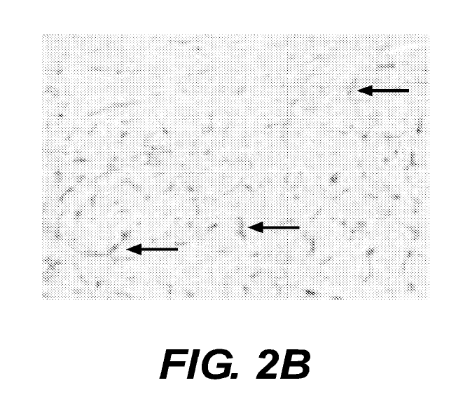

The target for G-202, as stated above, is PSMA, a carboxypeptidase that multiple studies have demonstrated is highly expressed by tumor vasculature within the majority of human tumors. For example, a series of studies over the last decade have documented that PSMA, besides being expressed almost universally by prostate cancer cells, is also uniquely expressed by tumor endothelial cells within the tumor vasculature of many tumor types. See, e.g., Israeli, R. S., et al., Cancer Res. 1994; 54: 1807-1811; Kawakami, M., et al., Cancer Res. 1997; 57:2321-2324; Minner, S., et al., Prostate 2011; 71:281-288. In contrast, PSMA is not expressed by the normal vasculature or the epithelium of most normal tissues. See, e.g., Silver, D. A., et al., Clin. Cancer Res. 1997; 3:81-85; Liu, H., et al., Cancer Res. 57, 3629-3634 (1997); Chang, S. S., et al., Clin. Cancer Res. 1999; 5:2674-2681; Chang, S. S., et al., Cancer Res. 1999; 59:3192-3198; Haffner, M. C., et al., Hum. Pathol. 2009; 40: 1754-1761. In this regard, Drs. Denmeade and Isaacs at Johns Hopkins University have used standard immunohistochemical staining of a tumor tissue array to demonstrate PSMA expression in tumor vasculature in ˜95% of HCC (N=42 cases) with no staining of endothelial cells in normal liver specimens (N=9 cases). Of the tumors sampled in this study, HCC demonstrated the highest level of PSMA staining in the tumor vasculature (see FIG. 2). See, e.g., Denmeade, S., et al., www.ScienceTranslationalMedicine.org, Vol. 4, Issue 140: 1-12 (2012).

Utilizing this data, G-202 was expected to have an impact on the progression of HCC. However, surprisingly, what was also observed in the treatment of patients diagnosed with HCC with G-202 was the tolerance of these patients to the potentially toxic effects of G-202. As is disclosed herein, patients having reduced liver function, for example, patients diagnosed with HCC and previously treated with sorafenib, would be expected to exhibit side effects associated with the administration of G-202. As is discussed above, G-202 is a prodrug utilizing a thapsigargin derivative and would be expected to exhibit some toxicity to non-cancer cells, and particularly to the liver as the liver is responsible for breaking down toxins in the body and drug metabolism.

Indeed, it is common practice to reduce the recommended dose of a chemotherapy agent, for example, sorafenib, or discontinue use once a patient begins so show signs of intolerance to the drug. While dose reduction is expected in all forms of cancer treatment, it is particularly expected in patients of liver cancer given the liver's function in the body and its role in drug metabolism. For these patients, already diminished liver function due to the effects of HCC is further exacerbated by the administration of a toxic chemotherapy agent. Thus, what is typically observed in HCC patients is a greater reduction in the dosage of, or an earlier discontinuation of, treatment relative to other forms of cancer due to the toxicity of the chemotherapeutic agent.

Therefore, it was expected in the studies discussed in the Examples herein that HCC patients would require dose reductions in the administration of G-202 given their compromised liver function. Unexpectedly, what was observed was that there was no need to reduce the amount of G-0202 administered to these patients or discontinue its use in any patients due to intolerance to the drug or its side effects. Thus, a relatively high dose of G-202 could be administered to patients, allowing for the more effective treatment of HCC, as compared to current protocols, such as treatment with sorafenib.

Administration of G-202

As stated above, the active compounds of the invention can be administered parenterally by injection or by gradual infusion over time. The prodrugs can be administered intravenously, intraperitoneally, intramuscularly, subcutaneously, intracavity, orally, or transdermally. Preferred methods for delivery of the active compounds of the invention include intravenous or subcutaneous administration. Other methods of administration, as well as dosing regimens, will be known to those skilled in the art.

The compositions of the present invention may be administered as a single dose or multiple doses. It may be infused for less than 1 hour, between 1 to 2 hours, or for 2 hours or longer. The treatment method may be performed once or repeated depending on the severity of the disease. Furthermore, the treatment may be reiterated upon recurrence of the disease.

The composition of the present invention may also be provided in an article of manufacture, or a “kit”. The article of manufacture comprises a container and a label or package insert on or associated with the container. Suitable containers include, for example, bottles, vials, syringes, blister pack, etc. The containers may be formed from a variety of materials such as glass or plastic. The container holds a compound or formulation thereof effective for treating the condition and may have a sterile access port (for example the container may be an intravenous solution bag or a vial having a stopper pierceable by a hypodermic injection needle). The label or package insert indicates that the composition is used for treating the condition of choice, such as cancer, and may contain warnings and instructions for administration.

The treatment according to the present invention may be supplemented with any other relevant treatment for epithelial cancers, for example prostate carcinoma and hepatocellular carcinoma. Such supplemental treatment may be given before, at the same time or after the administration of the composition of the invention and it may be given at frequencies normally used for such treatments. A suitable example of supplemental treatment is chemotherapy and the like. Surgical methods for treating epithelial tumor conditions may also be employed in combination with the methods of the present invention.

The methods of the invention are also directed towards the treatment of subjects with metastatic tumors. In some embodiments, the metastatic tumors are of epithelial origin. Carcinomas may metastasize to bone, as has been observed with breast cancer, and liver, as is sometimes the case with colon cancer. The methods of the invention are intended to treat metastatic tumors regardless of the site of the metastasis and/or the site of the primary tumor. In preferred embodiments, the metastases are of epithelial origin.

The invention will now be described in greater detail by reference to the following non-limiting examples.

### EXAMPLE 1

### Preparation of G-202 for Administration

The following provides a description of Phase I clinical trial studies of human cancer patients administered G-202 to determine the dosage of the drug to be used in Phase II. The following study supplies were provided to the clinical site for use in the study:
(a) Clear amber vials containing lyophilized G-202 Drug Product, stored at or below −20° C.
(b) Clear amber vials containing Sterile Propylene Glycol, stored at room temperature.
(c) Clear amber vials containing Sterile Polysorbate 20 in 0.9% Saline Solution, stored at room temperature.

Specific instructions for storage of G-202 and its diluents, reconstitution of G-202 and preparation of the final dosing solution were provided to the clinical site pharmacy. A volume of Sterile Propylene Glycol was used to rehydrate the lyophilized G-202 Drug Product. A volume of Sterile Polysorbate 20 in 0.9% Saline Solution was added immediately to the vial containing G-202 and Sterile Propylene Glycol. The mixture was allowed to mix and then introduced into an intravenous infusion bag containing normal saline, known as G-202 Dosing Solution, and was provided by the site pharmacy for administration to the patient. G-202 Dosing Solution was labeled with the study number of the patient for whom it is intended. The investigative staff confirmed this information and its relevancy to the intended patient.

Reconstituted G-202 is stable at room temperature for at least 24 hours. G-202 Dosing Solution is stable at room temperature for at least 24 hours.

### EXAMPLE 2

### A Study to Evaluate the Safety and Clinical Efficacy of G-202

G-202 was evaluated in a phase I trial G-202-001 to assess primarily the safety and secondarily the efficacy of G-202. The G-202-001 trial was divided into two components. The first component was designed to assess safety of various doses of the drug. Endpoints evaluated in the study included safety, tumor response rate, progression-free survival, and overall survival. Additional secondary endpoints included exploration of pharmacodynamic markers associated with various cancers. Assessments were routinely performed during therapy and following therapy if toxicity was the reason therapy was discontinued.

A total of 28 patients having various types of cancer were enrolled to this component of the G-202-001 study. All of the patients had advanced cancer and had received prior treatments for their cancer. The patients received G-202, which was administered at various dose levels, intravenously over 1 hour for up to 3 consecutive days of a 28-day cycle. Tested dose levels ranged from 1.2 mg/m² to 88 mg/m². It was determined that 66.8 mg/m² administered on Days 1, 2 and 3 of a 28-day cycle was the maximum tolerated dose and should be further explored to evaluate efficacy.

G-202 was well-tolerated by most patients with a manageable safety profile.

### EXAMPLE 3

### A Study to Evaluate the Safety and Clinical Efficacy of a Modified Dosing Regimen of G-202

Virtually all anti-cancer agents have the potential to cause infusion reactions, which are reactions that may be allergic or non-allergic in nature and not related to the known toxicity profile of the agent. Infusion reactions are usually thought of as hypersensitivity reactions, despite the absence of an allergic component in many cases. Because infusion reactions are not uncommon with anti-cancer agents given by intravenous infusion, a modified dosing regimen for G-202 was developed to reduce the likelihood of a patient having an infusion reaction. In the modified dosing regimen, the G-202 dose on Day 1 of each cycle is reduced. The G-202 dose on Day 1 of each 28-day cycle is 40 mg/m²; the G-202 dose level on Days 2 and 3 of each 28-day cycle is 66.8 mg/m². On all three days, G-202 is administered by intravenous infusion over 1 hour.

Fifteen (15) patients with various types of cancer were enrolled in an expansion group in trial G-202-001 to evaluate the modified dosing regimen. All of the patients had advanced cancer and had received prior treatments for their cancer. Endpoints evaluated in the expansion group included safety, tumor response rate, progression-free survival, and overall survival. Additional secondary endpoints included exploration of pharmacodynamic markers associated with various cancers. Assessments were routinely performed during therapy and following therapy if toxicity was the reason therapy was discontinued.

The occurrence of infusion-related reactions was decreased with the modified dosing regimen. The modified dosing regimen was determined to be suitable for further clinical studies.

Among the fifteen patients enrolled in the expansion group, five (5) patients were diagnosed with hepatocellular carcinoma. All five of these patients had advanced, unresectable HCC and had already had prior treatment with sorafenib for their cancer. The only FDA-approved therapy for patients with unresectable HCC is sorafenib tosylate, otherwise known as Nexavar® and/or by the chemical name: 4-(4-{3-[4-Chloro-3-(trifluoromethyl)phenyl]ureido}phenoxy)N2methylpyridine-2-carboxamide 4-methylbenzenesulfonate.

In a clinical trial of 602 patients with HCC and upon which FDA approval for Nexavar/sorafenib was granted, the average time to disease progression for patients with HCC after beginning sorafenib treatment is approximately six months (Llovet, J. M., et al, N Engl J Med 2008; 359:378-390). In patients with HCC who have taken sorafenib and go on to receive either no treatment or an ineffective treatment, the average time for HCC to progress is approximately 2.1 months (Finn, R. S., et al. Clin Cancer Res 2012; 18: 2090-2098; Yau, T., et al., Invest New Drugs 2012; 30(6):2384-90; Llovet, J. M., et al. International Liver Congress 2012, Abstract 1398).

The five patients with HCC enrolled in the expansion group had previously been treated with sorafenib and either experienced disease progression or unacceptable side effects. These patients were treated with G-202 under the following 28-day protocol:

Day 1: G-202 administered at about 40 mg/m²

Day 2: G-202 administered at about 66.8 mg/m²

Day 3: G-202 administered at about 66.8 mg/m²

The safety and effectiveness of G-202 on controlling or treating their disease was evaluated.

Of the five patients with HCC, two patients reached disease progression after two 28-day treatment cycles with G-202. Another patient reached disease progression after four 28-day treatment cycles with G-202. A fourth patient reached disease progression after twelve 28-day treatment cycles with G-202. The fifth patient had previously received sorafenib but experienced disease progression after only 3 months of treatment. The same patient experienced stable disease after treatment with G-202; the patient's disease stabilization persisted through nine 28-day cycles before the patient experienced a treatment delay due to an unrelated medical event (broken hip) and had to discontinue participation in the study; this patient's HCC appears to be stable and the patient continues to see no disease progression more than four months after ceasing treatment with G-202.

### EXAMPLE 4

### Additional Phase II Studies to Evaluate the Safety and Clinical Efficacy of G-202

A single-arm phase II trial of G-202, trial number G-202-003, in patients with progressive advanced hepatocellular carcinoma (HCC) has been implemented. Eligible patients are those who have progressed on or were intolerant of sorafenib therapy and have at least one measurable lesion.

Following study registration, patients begin single-agent therapy with G-202 which is to be administered intravenously on Days 1, 2, and 3 of each 28-day cycle. A safety run-in phase is planned to confirm the dose to be used for patients with HCC many of whom have compromised liver function due to their liver cancer and due secondarily to underlying liver cirrhosis and/or viral hepatitis.

The primary endpoint of this study is time to progression (TTP). Secondary endpoints include tumor response rate, progression-free survival, and overall survival. Identification of target and non-target lesions and assessment of treatment response and progression will be conducted according to the recommendations specified in the modified Response Criteria in Solid Tumors (mRECIST) for HCC. Assessments will be performed every 8 weeks during therapy and following therapy if toxicity was the reason therapy was discontinued. Patients' vital status needed for secondary survival endpoints will continue to be reported after disease progression.

Additional secondary endpoints include exploration of pharmacodynamic markers using three approaches:
Alpha fetoprotein level changes will be assessed in all patients.
Formalin-fixed paraffin-embedded (FFPE) tissue from a previous resection or core biopsy of primary or metastatic tumor may be obtained for assessment of changes in PSMA expression and apoptotic index. If a core biopsy is performed to confirm HCC diagnosis, fresh frozen and FFPE samples will be required at baseline and, for consenting patients, following treatment in Cycle 2.
Investigators at sites with the technical capability to perform dynamic contrast-enhanced magnetic resonance imaging (DCE-MRI) may perform DCE-MRIs (for consenting patients) at baseline and following treatment in Cycle 2 to assess changes in tumor vasculature and blood flow.

The expected sample size for the study is a minimum of 29 and maximum of 35. Accrual is expected to be 2-3 patients per month.

### EXAMPLE 5

### Preliminary Results of Additional Phase II Studies to Evaluate the Safety and Clinical Efficacy of G-202

Four patients have been evaluated under the study parameters described in Example 4. Each patient showed disease progression after sorafenib or was unable to tolerate sorafenib.

The first three patients in this trial were treated with G-202 under the following 28-day protocol:

Day 1: G-202 administered at about 40 mg/m²

Day 2: G-202 administered at about 40 mg/m²

Day 3: G-202 administered at about 40 mg/m²

One patient was treated with G-202 and additional patients are being enrolled for treatment under the following 28-day protocol:

Day 1: G-202 administered at about 40 mg/m²

Day 2: G-202 administered at about 66.8 mg/m²

Day 3: G-202 administered at about 66.8 mg/m²

Safety data from the first three patients showed that G-202 is tolerated in this patient population, i.e., those with advanced liver cancer and compromised liver function.

Two patients show no improvement on G-202 therapy and disease progression was reached after no more than two cycles. Of the two other patients, one patient exhibited deterioration in liver function after five 28-day treatment cycles with G-202 and was removed from the study; in the opinion of the treating physician, the deterioration in liver function was due to the patient's cancer and was not a side effect of G-202. Imaging assessments showed that the patient's cancer had not grown and he would have continued to receive G-202 if his liver function had not deteriorated. The other patient continues to receive G-202 treatment; he currently has completed five 28-day treatment cycles, has begun a sixth cycle and shows no signs of disease progression.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps of the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Other embodiments are set forth within the following claims.

What is claimed is:

1. A method of treating a subject having a tumor comprising administering an effective amount of the prodrug G-202 to said subject.

2. The method of claim 1, wherein said effective amount of said prodrug is administered for at least one day of a 28-day cycle.

3. The method of claim 2, wherein said effective amount of said prodrug comprises administering at least about 40 mg/m² of said prodrug.

4. The method of claim 3, wherein said effective amount of said prodrug comprises administering at least about 60 mg/m² of said prodrug.

5. The method of claim 4, wherein said effective amount of said prodrug comprises administering at least about 80 mg/m² of said prodrug.

6. The method of claim 2, wherein said effective amount of said prodrug is administered for at least two consecutive days.

7. The method of claim 6, wherein said at least two consecutive days occurs at the beginning of a 28-day cycle.

8. The method of claim 7, wherein said effective amount of said prodrug comprises administering about 40 mg/m² of said prodrug on day 1, and about 66.8 mg/m² of said prodrug on day 2.

9. The method of claim 7, wherein said effective amount of said prodrug comprises administering about 40 mg/m² of said prodrug on days 1 and 2.

10. The method of claim 6, wherein said effective amount of said prodrug is administered for at least three consecutive days.

11. The method of claim 10, wherein said at least three consecutive days occurs at the beginning of a 28-day cycle.

12. The method of claim 11, wherein said effective amount of said prodrug comprises administering about 40 mg/m² of said prodrug on day 1, and about 66.8 mg/m² of said prodrug on each of days 2 and 3.

13. The method of claim 11, wherein said effective amount of said prodrug comprises administering about 40 mg/m² of said prodrug on days 1, 2 and 3.

14. The method of claim 1, wherein said tumor is selected from the group consisting of prostate cancer, hepatocellular carcinoma and glioblastoma.

15. The method of claim 2, wherein said 28-day cycle is repeated at least once.

16. The method of claim 2, wherein administration is selected from the group consisting of intravenous, intramuscular, subcutaneous, implantable pump, continuous infusion, liposomal and oral administration.

17. The method of claim 16, wherein said prodrug is administered via infusion.

18. The method of claim 16, wherein said prodrug is administered via injection.

19. The method of claim 16, wherein said prodrug is administered in combination with chemotherapy, surgery, other procedure, other anti-cancer agent(s) or other therapeutic methods.

20. The method of claim 19, wherein said prodrug is administered prior to surgery.

21. The method of claim 20, wherein said prodrug is administered for at least two 28-day cycles.

22. A method of preventing the spread of, stabilizing or reducing a tumor comprising administering and effective amount of the prodrug G-202 to a subject having a tumor.

23. The method of claim 22, wherein said effective amount of said prodrug is administered for at least one day of a 28-day cycle.

24. The method of claim 23, wherein said effective amount of said prodrug comprises administering at least about 40 mg/m² of said prodrug.

25. The method of claim 24, wherein said effective amount of said prodrug comprises administering at least about 60 mg/m² of said prodrug.

26. The method of claim 25, wherein said effective amount of said prodrug comprises administering at least about 80 mg/m² of said prodrug.

27. The method of claim 22, wherein said effective amount of said prodrug is administered for at least two consecutive days.

28. The method of claim 27, wherein said at least two consecutive days occurs at the beginning of a 28-day cycle.

29. The method of claim 28, wherein said effective amount of said prodrug comprises administering about 40 mg/m² of said prodrug on day 1, and about 66.8 mg/m² of said prodrug on day 2.

30. The method of claim 28, wherein said effective amount of said prodrug comprises administering about 40 mg/m² of said prodrug on days 1 and 2.

31. The method of claim 27, wherein said effective of said prodrug amount is administered for at least three consecutive days.

32. The method of claim 31, wherein said at least three consecutive days occurs at the beginning of a 28-day cycle.

33. The method of claim 32, wherein said effective amount of said prodrug comprises administering about 40 mg/m² of said prodrug on day 1, and about 66.8 mg/m² of said prodrug on each of days 2 and 3.

34. The method of claim 32, wherein said effective amount of said prodrug comprises administering about 40 mg/m² of said prodrug on days 1, 2 and 3.

35. The method of claim 22, wherein said tumor is selected from the group consisting of prostate cancer, hepatocellular carcinoma and glioblastoma.

36. The method of claim 23, wherein said 28-day cycle is repeated at least once.

37. The method of claim 23, wherein administration is selected from the group consisting of intravenous, intramuscular, subcutaneous, implantable pump, continuous infusion, liposomal and oral administration.

38. The method of claim 37, wherein said prodrug is administered via infusion.

39. The method of claim 37, wherein said prodrug is administered via injection.

40. The method of claim 37, wherein said prodrug is administered in combination with chemotherapy, surgery, other procedure, other anti-cancer agent(s) or other therapeutic methods.

41. The method of claim 40, wherein said prodrug is administered prior to surgery.

42. The method of claim 41, wherein said prodrug is administered for at least two 28-day cycles.

43. A method for treating prostate cancer, hepatocellular carcinoma or glioblastoma in a patient comprising administering an effective amount of the prodrug G-202 to said patient.

44. The method of claim 43, wherein said effective amount of said prodrug is administered for at least one day of a 28-day cycle.

45. The method of claim 44, wherein said effective amount of said prodrug comprises administering at least about 40 mg/m² of said prodrug.

46. The method of claim 45, wherein said effective amount of said prodrug comprises administering at least 60 about mg/m² of said prodrug.

47. The method of claim 46, wherein said effective amount of said prodrug comprises administering at least 80 about mg/m² of said prodrug.

48. The method of claim 44, wherein said effective amount of said prodrug is administered for at least two consecutive days.

49. The method of claim 48, wherein said at least two consecutive days occurs at the beginning of a 28-day cycle.

50. The method of claim 49, wherein said effective amount of said prodrug comprises administering about 40 mg/m² of said prodrug on day 1, and about 66.8 mg/m² of said prodrug on day 2.

51. The method of claim 49, wherein said effective amount of said prodrug comprises administering about 40 mg/m² of said prodrug on days 1 and 2.

52. The method of claim 44, wherein said effective amount of said prodrug is administered for at least three consecutive days.

53. The method of claim 52, wherein said at least three consecutive days occurs at the beginning of a 28-day cycle.

54. The method of claim 53, wherein said effective amount of said prodrug comprises administering about 40 mg/m² of said prodrug on day 1, and about 66.8 mg/m² of said prodrug on each of days 2 and 3.

55. The method of claim 53, wherein said effective amount of said prodrug comprises administering about 40 mg/m² of said prodrug on days 1, 2 and 3.

56. The method of claim 44, wherein said 28-day cycle is repeated at least once.

57. The method of claim 44, wherein administration is selected from the group consisting of intravenous, intramuscular, subcutaneous, implantable pump, continuous infusion, liposomal and oral administration.

58. The method of claim 57, wherein said prodrug is administered via infusion.

59. The method of claim 57, wherein said prodrug is administered via injection.

60. The method of claim 57, wherein said prodrug is administered in combination with chemotherapy, surgery, other procedure, other anti-cancer agent(s) or other therapeutic methods.

61. The method of claim 60, wherein said prodrug is administered prior to surgery.

62. The method of claim 61, wherein said prodrug is administered for at least two 28-day cycles.

63. A kit comprising an effective amount of G-202.

64. The kit of claim 63 wherein G-202 is lyophilized.
